(19) United States
(12) Patent Application Publication
(10) Pub. No.: US 2006/0094937 A1
(43) Pub. Date: May 4, 2006

(54) Monitoring apparatus of arterial pulses and method for the same

(75) Inventors: Igor Yakovlevich Immoreev, Moscow (RU); Sergey Vital'evich Samkov, Moscow region (RU); Teh-Ho Tao, Hsinchu City (TW)

Correspondence Address: John S. Egbert;Harrison & Egbert, 7th Floor, 412 Main Street, Houston, TX, 77002, US

(73) Assignee: MARCOTT INTERNATIONAL INVESTMENT, Co. Ltd., Taipei City (TW)

(21) Appl. No.: 10/979,990

(22) Filed: Nov. 3, 2004

(51) Int. Cl. A61B 5/00

(52) U.S. Cl. 600/301

(57) ABSTRACT

A monitoring apparatus includes an antenna board including a transmission antenna for transmitting an ultra wideband electromagnetic wave to an arterial blood vessel and a reception antenna for receiving the ultra wideband electromagnetic wave scattered by the arterial blood vessel, an analog board with a plurality of electronic devices for acquiring analog signals representing the arterial pluses of the arterial blood vessel, a digital board with a plurality of electronic devices for digitalizing the analog signal, and a display device for showing the arterial pulses. The method for acquiring arterial pulses first radiates an ultra wideband electromagnetic wave to an arterial blood vessel, and measures the phase difference between the ultra wideband electromagnetic wave scattered by the arterial blood vessel and the reference ultra wideband electromagnetic wave. Finally, the arterial pulses are acquired based on the variation of the phase difference.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention relates to a monitoring apparatus of arterial pulses and method for the same, and more particularly to a monitoring apparatus of arterial pulses using a non-contact measurement scheme in which an ultra wideband (UWB) electromagnetic wave is employed to detect the movement of an arterial blood vessel and method for acquiring arterial pulses using for the same.

### BACKGROUND OF THE INVENTION

The purpose of monitoring arterial pulses is to alert patient or his/her caregivers of abnormal conditions of arterial circulatory system, which may lead to heart failure. Conventional technologies for monitoring patient's arterial pulses most commonly employ piezoelectric pressure sensors to obtain signal on body sites where arterial blood vessels are located. U.S. Pat. No. 4,489,731 discloses a wrist-worn device using a piezoelectric crystal as the detector for sonic vibrations caused by arterial pulses. U.S. Pat. No. 5,807,267 discloses a wrist-worn device using two piezoelectric sensors to reduce the noises caused by body motion. U.S. Pat. No. 5,795,300 discloses a device containing piezoelectric sensing elements preferably positioned over the radial artery in the subpollex depression parallel to the tendon cord bundles on the volar surface of the wrist so as to maximize the signal-to-noise ratio. During operation, these devices must be pressed against the patient's skin with external pressure so that signal with good quality can be obtained. However, it may cause the patient's discomfort in long-term operation due to prolong compression of the skin tissue. Furthermore, skin irritation and sweating may be induced, which results in signal-to-noise ratio degradation and signal baseline drift.

The other commonly used method employs optical sensors to acquire arterial pulse signal due to the light transmission through pulsating vascular bed. Although the sensor does not require close contact with the skin, it can only be used effectively on peripheral extremities where enough light passing through vascular bed can be received by the light detector. U.S. Pat. No. 5,573,012 uses the ultra wideband (UWB) electromagnetic means to detect movements of heart, lung and vocal cord. The device is designed to use the non-acoustic pulse radar monitoring employed in the repetitive mode, whereby a large number of reflected pulses are averaged. It incorporates a time-gate scheme of which time gating pulses are generated by a range delay generator. The time gating pulses cause the receive path to selectively conduct pulses reflected from the body parts and received by a receive antenna. The device converts the detected voltage into an audible signal using both amplitude modulation and Doppler effect. The device is designed for remote operation and thus cannot be attached to the patient under examination. Therefore, it is not suitable for portable long-term monitoring purpose.

In view of the aforementioned problems, an arterial pulse monitoring apparatus based on a non-contact measurement scheme, which can be worn by patients and provide the characteristics of long-term stable operation, small size and low cost is needed.

### BRIEF SUMMARY OF THE INVENTION

The objective of the present invention is to provide a monitoring apparatus of arterial pulses using a non-contact measurement scheme in which ultra wideband electromagnetic wave is employed to detect movement of an arterial blood vessel and method for acquiring arterial pulses for the same.

In order to achieve the above-mentioned objective, and avoid the problems of the prior art, the present invention provides a monitoring apparatus of arterial pulses using a non-contact measurement scheme and method for the same. The monitoring apparatus comprises an antenna board including a transmission antenna for radiating ultra wideband electromagnetic wave to an arterial blood vessel and a reception antenna for receiving the ultra wideband electromagnetic wave scattered by the arterial blood vessel, an analog board with a plurality of electronic devices for acquiring analog signals representing the arterial pluses of the arterial blood vessel, a digital board with a plurality of electronic devices for digitalizing the analog signal, and a display device for showing the arterial pulses.

The present method for acquiring arterial pulses first radiates a series of ultra wideband electromagnetic pulses (probing pulses) to an arterial blood vessel. Secondly, the time variation of phase differences between the probing pulses scattered by an arterial blood vessel and the radiated probing pulses are measured according to following equations:

$$Δφ = Δφ 1 - Δφ 2 = \frac{4 π \cdot \sqrt{ɛ} \cdot f}{c} ( {D}_{1} - {D}_{2} )$$

$${D}_{2} = {D}_{1} - {VT}_{a}$$

Wherein D₁ represents the distance between the arterial blood vessel and the skin surface, V represents the radial velocity of the arterial blood vessel toward where the probing pulses are radiated, Tₐ represents the time interval as the arterial blood vessel moves from D₁ to D₂, f represents the repetition frequency of the probing pulses, c represents velocity of light and e represents the relative dielectric constant of the human skin tissue. Finally, the movement of an arterial vessel D₁-D₂ is detected based on its linear relationship to the phase difference Δφ.

### BRIEF DESCRIPTION OF THE SEVERAL VIEWS OF THE DRAWINGS

Other objectives and advantages of the present invention will become apparent upon reading the following descriptions and upon reference to the accompanying drawings in which:

FIGS. 1(a), 1(b) and 1(c) show the present monitoring apparatus attached onto a human body sits where movements of arterial blood vessels can be detected;

FIGS. 2(a) and 2(b) show the monitoring apparatus according to the present invention;

FIG. 3(a) shows the time diagram of the probing pulse;

FIG. 3(b) shows the frequency spectrum of the radiated probing pulse;

FIG. 4 is a functional block diagram of the monitoring apparatus according to the present invention;

FIGS. 5(a), 5(b), 5(c) and 5(d) are circuit diagrams of the analog board according to the present invention;

FIG. 6(a) and 6(b) illustrate the operation of the balance mixer according to the present invention;

FIG. 7(a) shows the timing relationship of the received probing pulses and the reference pulses at the balance mixer; FIG. 7(b) shows time diagrams of signals at the output of the balance mixer and of the first low-pass filter;

FIG. 8(a) shows the signal time diagram at the output of the first low-pass filter when the movement of the arterial blood vessel is not detected;

FIG. 8(b) shows the time diagram of the signal at the output of the first low-pass filter when movement of arterial blood vessel is detected;

FIG. 9 shows the frequency spectrum of the balance mixer's output signal according to the present invention;

FIG. 10 shows both the signal spectrum envelope inputting to the transmission antenna and antenna's frequency performance according to the present invention;

FIG. 11 shows a circuit diagram of the digital board according to the present invention;

FIG. 12(a) shows a loop antenna suitable for the monitoring apparatus according to the present invention;

FIG. 12(b) shows a bow-tie antenna suitable for the monitoring apparatus according to the present invention;

FIG. 12(c) shows a terminating half-wave antenna suitable for the monitoring apparatus according to the present invention;

FIG. 12(d) shows a spiral antenna suitable for the monitoring apparatus according to the present invention;

FIGS. 13(a) and FIG. 13(b) show the comparison of radial arterial signal waveform using the present monitoring apparatus and the electrocardiogram signal waveform.

### DETAILED DESCRIPTION OF THE INVENTION

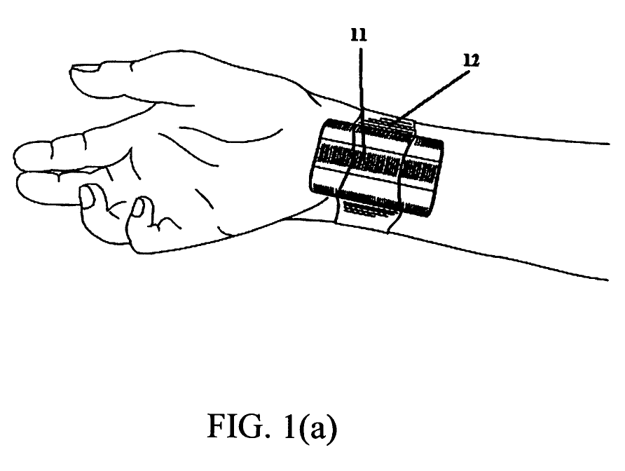

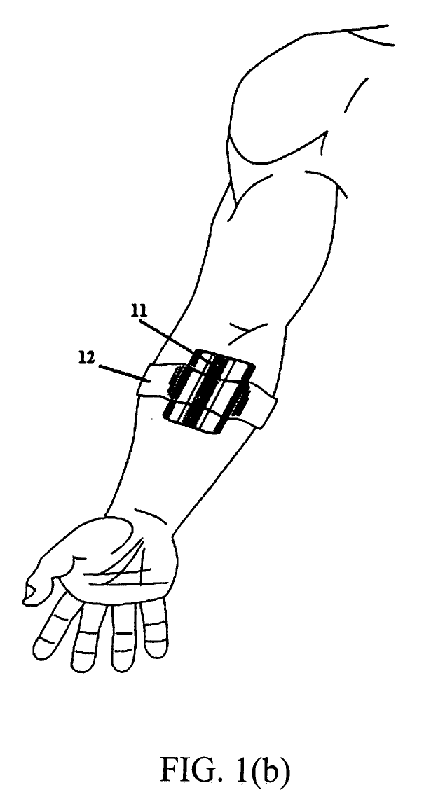

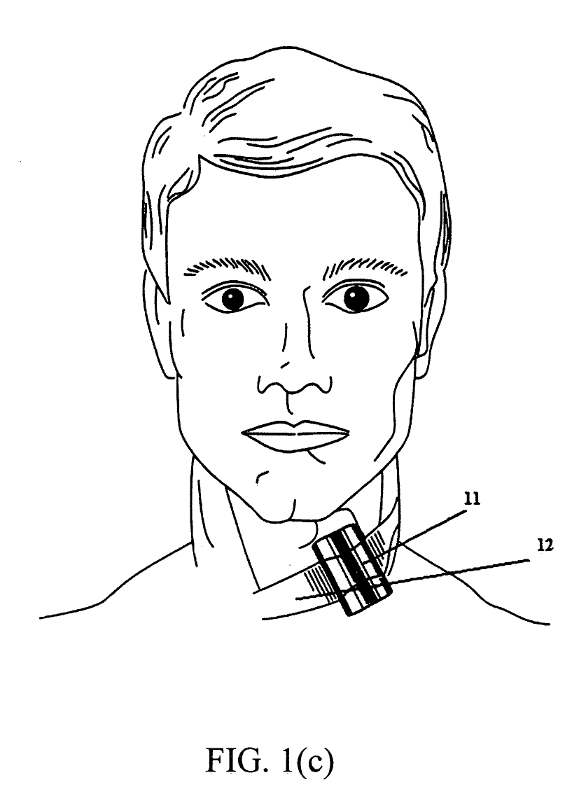

FIGS. 1(a), 1(b) and 1(c) show the present monitoring apparatus 10 attached onto human body sites where movements of arterial blood vessels can be detected. The strap 12 is used for attaching the monitoring apparatus 10 onto body sites without exerting pressure on the skin of the user.

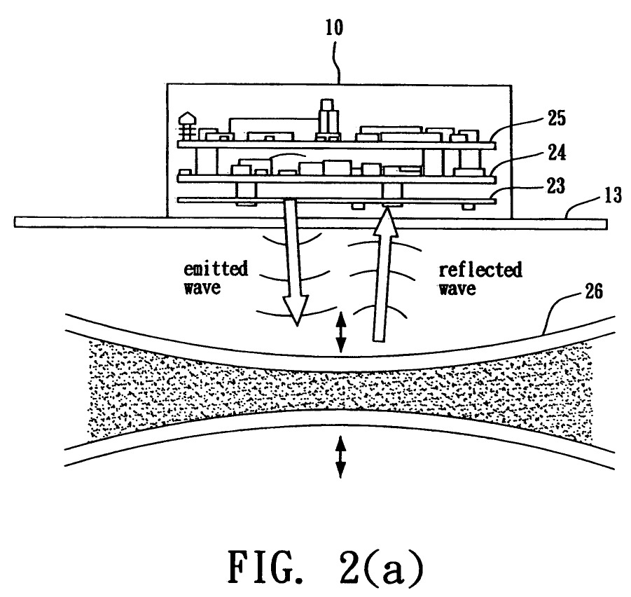

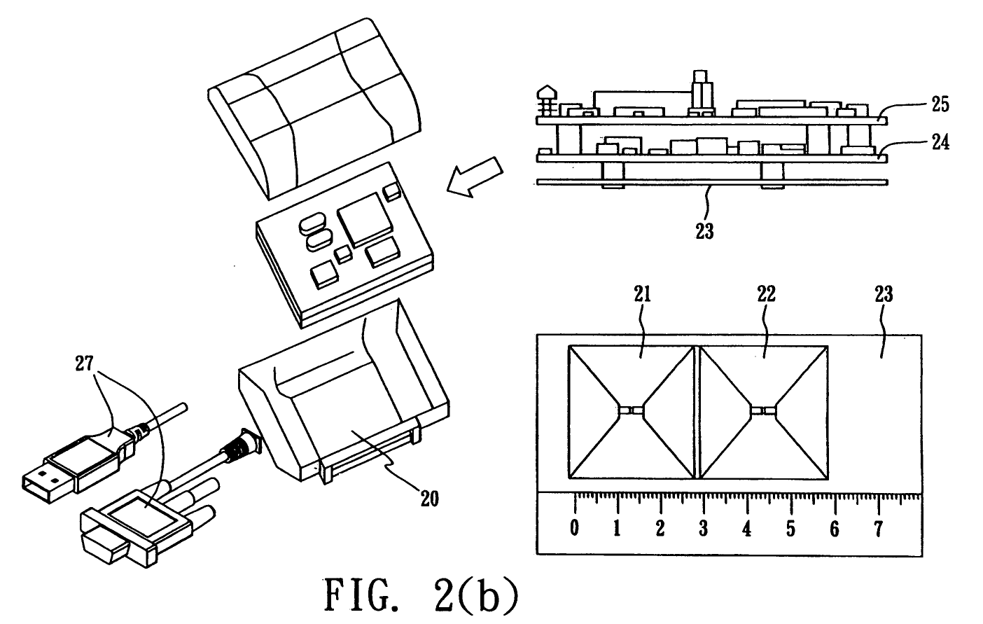

FIGS. 2(a) and 2(b) show the monitoring apparatus 10 according to the present invention. As shown in FIG. 2(a), the monitoring apparatus 10 comprises an antenna board 23, an analog board 24 and a digital board 25. As shown in FIG. 2(b), the antenna board 23 comprises a transmission antenna 21 and a reception antenna 22 from which a series of probing pulses are emitted and received through a membrane 20. The transmission antenna 21 and the reception antenna 22 are both in the form of bow-tie antenna. Due to the nature of the non-contact measurement scheme, the membrane 20 does not serve the purpose of signal energy conversion but acts as a part of the enclosure for the monitoring apparatus 10. Therefore, its material property should not cause attenuation of the energy of the probing pulses. The membrane 20 is preferably made from polymeric materials such as silicone rubber or polycarbonate with a thickness from 0.2 to 0.5 mm.

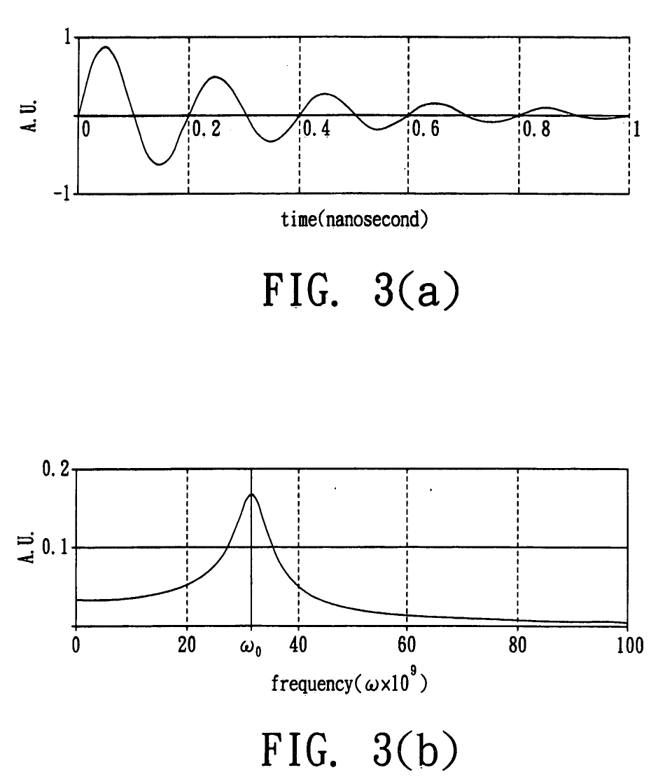

FIG. 3(a) shows the time diagram of the probing pulse. Each probing pulse consists of damped sinusoidal oscillations with its resonance frequency determined by the physical size of the transmission antenna. FIG. 3(b) shows the frequency spectrum of the probing pulse. The center frequency and the bandwidth are determined by the duration of the damped sinusoidal oscillations. When the duration of the damped sinusoidal oscillations approaches zero (ideal shape of an impulse), the center frequency and the bandwidth of the frequency spectrum approaches infinity.

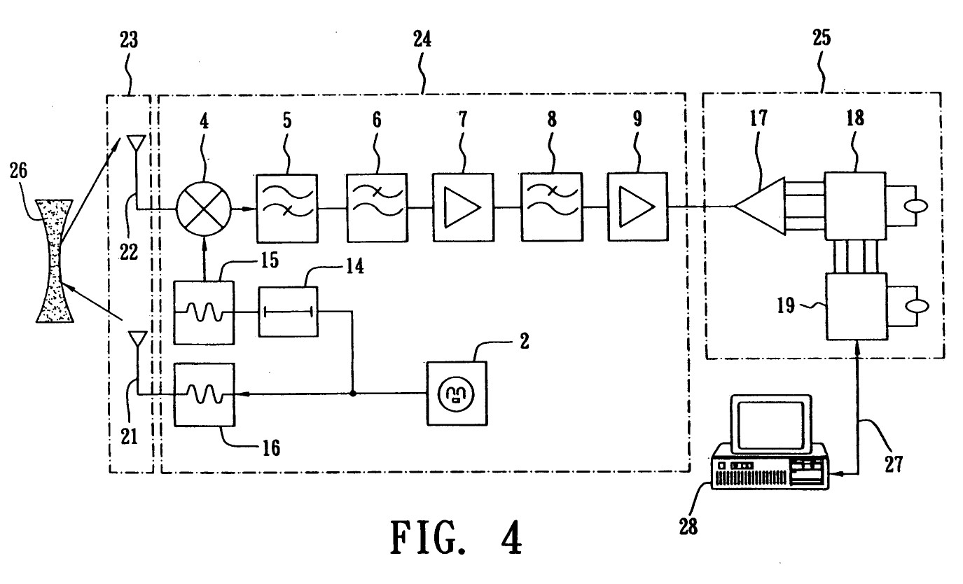

FIG. 4 is a functional block diagram of the monitoring apparatus 10 according to the present invention. The transmission antenna 21 and the reception antenna 22 are positioned on the antenna board 23, and both are performed on the basis of broadband dipole antenna (Bow-tie antenna). The analog board 24 comprises a balance mixer 4 connected to the reception antenna 22, a high-pass filter 5 connected to the mixer 4, a first low-pass filter 6 connected to the high-pass filter 5, a first amplifier 7 connected to the first low-pass filter 6, a second low-pass filter 8 connected to the first amplifier 7, a second amplifier 9 connected to the second low-pass filter 8, a first pulse generator 15 connected to the balance mixer 4, a delay line 14 connected to the first pulse generator 15, a second pulse generator 16 connected to the transmission antenna 21, and a clock generator 2 connected to the second pulse generator 16 and the delay line 14. The digital board 25 comprises a microcontroller 18 with an embedded analog-to-digital converter 17 and universal asynchronous transceiver 19.

Analog signal processing circuits on the analog board 24 separate a signal representing movement of the arterial blood vessel 26 from other spectral components due to scatter from tissue surrounding the arterial blood vessel 26. The detected signals are then digitized using the microcontroller 18 and transmitted using the transceiver 19 in the digital board 25. The digitized signals can be transmitted using either RS232 or USB cable 27 to an external data processing and display unit 28.

The UWB electromagnetic wave is selected due to following advantages:

1. Reduction in the spectral density of emission power. This makes it possible to lower the level of electromagnetic emission, which influences both doctor and patient, as well as the level of electromagnetic interference with other hospital equipments.

2. Simultaneous achievement of non-contact measurement and reduction in the device's overall sizes.

3. Increase in the device's immunity to external interferences and improvement in the reliability of measurement.

When constructing the present monitoring apparatus (a UWB radar), as when constructing conventional narrow-band radars, the property of electromagnetic wave to be scattered from a boundary of two media with different parameters is used, which is well-known from the general theory. The electromagnetic pulses radiated by radar are scattered by a moving object. In this case, the oscillation frequency f within the scattered pulses changes owing to Doppler effect. As frequency variation leads to variations of oscillation period T with the number of oscillation in the scattered pulses remains the same. Consequently the duration (τ) of the scattered pulses is changed. Due to the same effect, the repetition frequency of the electromagnetic pulses scattered by the object Fᵣ and, correspondingly, the repetition period Tᵣ also changes simultaneously. The sign of these variations depends on the direction of target movement relative to the radar and the variation value depends on the object's radial velocity.

Nevertheless, it should be noted that the usage of these effects in UWB radars, which are intended for detecting and measuring parameters of moving targets, has some specific features. In general case, to separate such a signal, variations of all three parameters of the pulse sequence mentioned above can be used. However, the oscillation frequency variations within a single scattered pulse are rather small because the duration of a single scattered pulse is very short. For example, for pulse duration τ=0.2 nanosecond, it does not contain one period of oscillation with frequency of 1 GHz. Therefore it is impossible to determine variation of frequency within a single scattered pulse using conventional filtering techniques.

On the contrary, it is possible to make an attempt to measure the phase difference, which appears between the series of scattered and the series of radiated pulses. The phase difference can be estimated as follows. When the object is moving towards the radar with a radial velocity V, the repetition period of the scattered pulses changes and becomes

$${T}_{0} = T ( 1 - \frac{V}{c} ) \qquad ( 1 )$$

Where c is the velocity of light. The phase difference and peculiarities of its variations during target movement can be determined as follows. If the instant value of the phase of the series of radiated pulses is φᵤ(t)=2πft, where f represents the repetition frequency of the radiated pulses, then the instant phase value for the series of pulses scattered by a stationary object located at a distance R is equal to:

$${φ}_{o} ( t ) = {φ}_{u} ( t ) + 2 π \cdot f \frac{2 R}{c} = 2 π \cdot f ( t + \frac{2 R}{c} ) \qquad ( 2 )$$

The phase difference between radiated and scattered pulses can be expressed as follows:

$$Δφ = {φ}_{u} ( t ) - {φ}_{o} ( t ) = - 4 π \cdot f \frac{R}{c} \qquad ( 3 )$$

From Eq.(3), the phase difference between the series of radiated and the series of scattered pulses due to movement of an arterial blood vessel can be derived as follows. If the arterial blood vessel is located at a distance R₁=Dₒ+D₁, where D₀ represents the distance between the surfaces of the antenna board 23 and that of the skin 13, D₁ represents the distance between the skin 13 and the arterial blood vessel 26, then the instant phase of the series of scattered pulses is:

$${φ}_{o 1} ( t ) = {φ}_{u} ( t ) + 2 π \cdot f \frac{2 ( {D}_{0} + {D}_{1} \sqrt{ɛ} )}{c} = 2 π \cdot f ( t + \frac{2 ( {D}_{0} + {D}_{1} \sqrt{ɛ} )}{c} ) \qquad ( 4 )$$

The phase difference between radiated and scattered pulses will be:

$${Δφ}_{1} = {φ}_{u} ( t ) - {φ}_{o 1} ( t ) = - 4 π \cdot f \frac{( {D}_{0} + {D}_{1} \sqrt{ɛ} )}{c} \qquad ( 5 )$$

If the arterial vessel moves to a distance R₂=D₀+D₂ from the radar, then the instance phase of the series of scattered pulses is:

$${φ}_{o 2} ( t ) = {φ}_{u} ( t ) + 2 π \cdot f \frac{2 ( {D}_{0} + {D}_{2} \sqrt{ɛ} )}{c} = 2 π \cdot f ( t + \frac{2 ( {D}_{0} + {D}_{2} \sqrt{ɛ} )}{c} ) \qquad ( 6 )$$

The phase difference between radiated and scattered pulses will have another value:

$${Δφ}_{2} = {φ}_{u} ( t ) - {φ}_{o 2} ( t ) = - 4 π \cdot f \frac{( {D}_{0} + {D}_{2} \sqrt{ɛ} )}{c} \qquad ( 7 )$$

Subtracting Eq.(7) from Eq.(5), the variation of the phase difference caused by the movement of arterial blood vessel is obtained, which is:

$$Δφ ( t ) = {Δφ}_{1} - {Δφ}_{2} = - \frac{4 π \cdot f \cdot \sqrt{ɛ}}{c} ( {D}_{1} - {D}_{2} ) = - \frac{4 π \cdot f \cdot \sqrt{ɛ}}{c} {VT}_{a} \qquad ( 8 )$$

Consequently, the phase difference Δφ varies from period to period and this variation depends on the velocity V and the period of oscillation Tₐ of the arterial blood vessel movement. With the repetition frequency of the radiated pulses f=10 MHz, ε=40, and an arterial blood vessel movement D₁-D₂=2 mm, the variation of the phase difference Δφ=0.3 degree is obtained, which allows the detection of phase difference using conventional phase measurement devices.

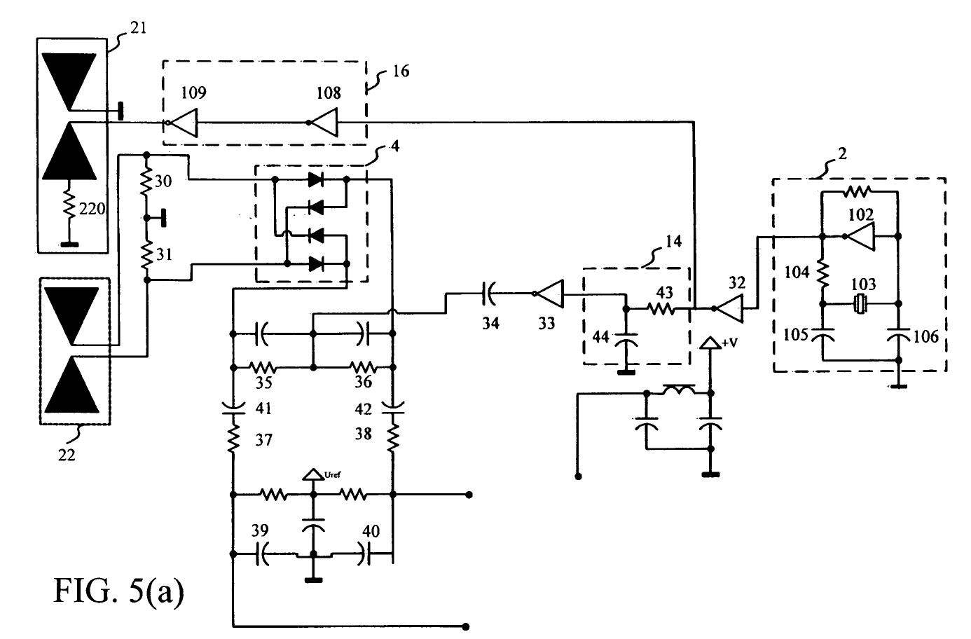

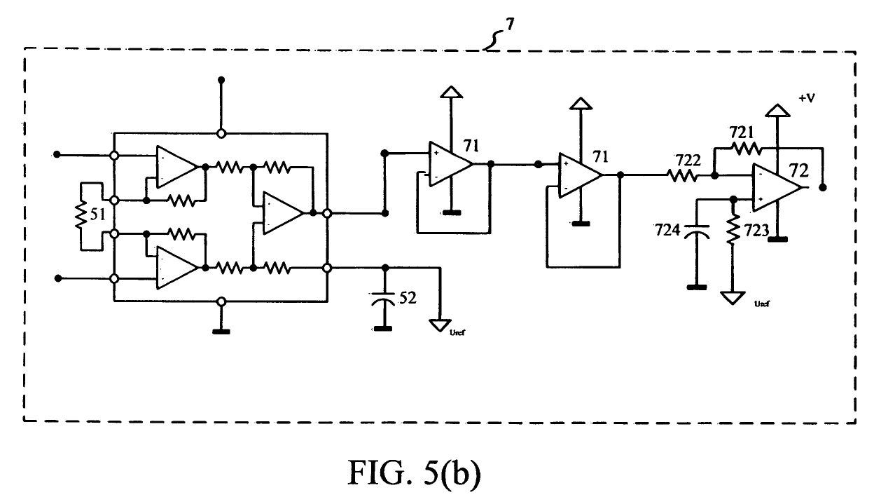

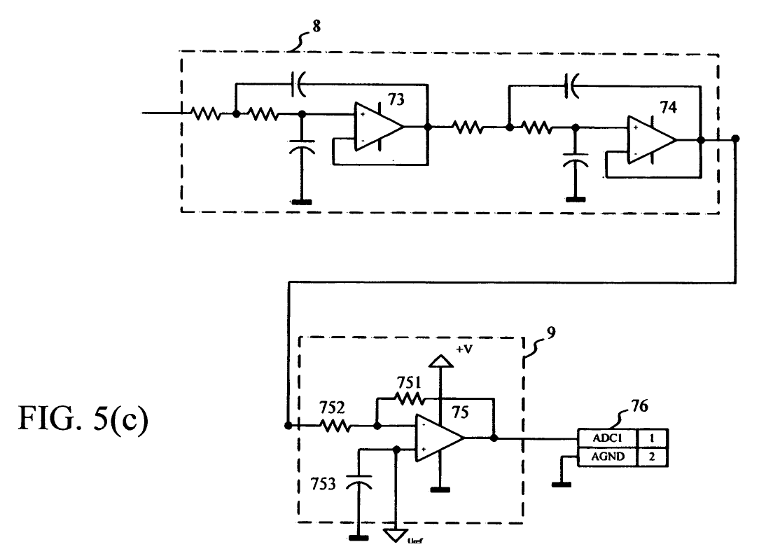

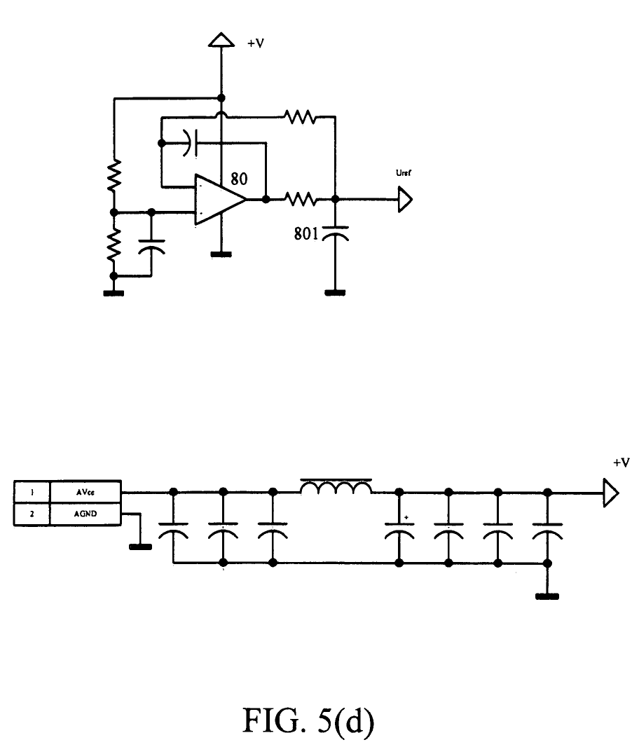

The operation of the monitoring apparatus 10 will be described in detail below. FIGS. 5(a), 5(b), 5(c) and 5(d) are circuit diagrams of the analog board 24 according to the present invention. The clock generator 2, realized on the logic inverter 102, produces square pulses and synchronizes the operation of the analog signal processing circuits on the analog board 24. The timing accuracy of the clock generator 2 is determined by the quartz crystal 103. Low cost crystals are available with an accuracy of ±30 ppm. Resistor 104 buffers the quartz crystal 103 from sharp logic transitions and prevents spurious oscillation modes. The combination of capacitors 105 and 106 forms an approximate load capacitance as specified for the quartz crystal 103. The resistor 104 provided a negative resistive feedback to bias the inverter 102 at its threshold (on average) and AC feedback through the quartz crystal 103 to control the oscillating frequency.

The second pulse generator 16 (a shaper of transmitter's probing pulse) consisting of logic inverters 108 and 109 is connected to transmission antenna 21. Pull-up resistor 220 (50 Ohm) is connected to the edge of the one vibrator of transmission antenna 21 to reduce duration of oscillations (“ring”) at the transmission antenna 21. The clock signal enters the receiver circuits via a buffer, realized on a logic inverter 32, which reduces influence of receiver to the operation of transmitting circuits and the clock generator 2. Reference pulses are formed from delayed clock signal from the clock generator 2 and go to the junction of the resistors 35 and 36 after they are shaped into short pulses by the first pulse generator 15 consisting of logic inverter 33, capacitor 34 and resistors 35, 36. The purpose of the delay line 14 is to match the timing between the radiated probing pulses and the reference pulses so that their phase differences can be correctly measured at the balance mixer 4. The time delay of the reference pulses is determined according to the following formula:

$${T}_{del} = 2 ( \frac{{D}_{0}}{C} + \frac{{D}_{1}}{C} \cdot \sqrt{ɛ} ) \qquad ( 9 )$$

Where D₀ represents the distance between the surface of the antenna board 23 and the skin 13, D₁ represents the distance between the skin 13 and the arterial blood vessel 26, C represents the velocity of light, and ε(≅=40) represents the relative dielectric constant of human skin tissue. As shown in FIG. 5(a), T_{del}=1.204 RC, where RC is the product of the resistance of the resistor 43 and the capacitance of the capacitor 44, which consists of the delay line 14.

Accepted by the reception antenna, the probing pulses proceed to the input contacts of the balance mixer 4. The resistors 30 and 31 are matched loads for the symmetric reception antenna 22.

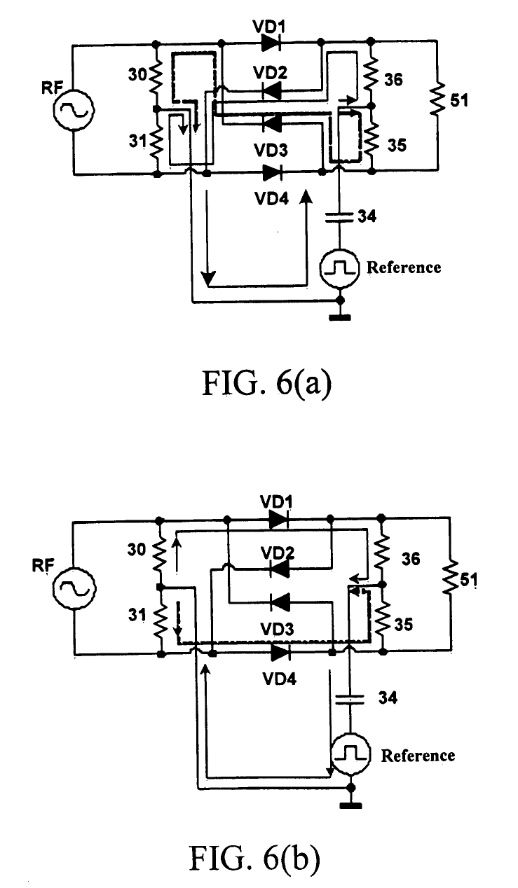

FIG. 6(a) and 6(b) illustrate the operation of the balance mixer 4. During the positive half-period of the reference pulse, the diodes VD2, VD3 are conducting and the received probing pulse proceeds to the output of the balance mixer as shown in FIG. 6(a). During the negative half-period of reference pulse (FIG. 6(b)), the diodes VD1, VD4 are conducting and the probing pulse proceeds to the output of the balance mixer with a phase shift of 180 degrees. Accordingly the output voltage of the balance mixer 4 is defined by the following expression:
U_{LOAD}=R(I_{VD1}−I_{VD4})+R(I_{VD2}−I_{VD3}) (10)

Where R represents the resistance of the resistors 35 and 36. The voltage-current characteristic of the diode can be approximated by a polynomial of the third degree I_{VD}=a₀+a₁U+a₂U²+a₃U³. Substituting this expression into equation (10), the following is obtained:

$$\begin{matrix}R ( {I}_{VD 1} - {I}_{VD 4} ) = 2 R ( {a}_{1} \frac{{U}_{RF}}{2} + 2 {a}_{2} \frac{{U}_{RF}}{2} {U}_{LO} + {a}_{3} \frac{{U}_{RF}^{3}}{2} + 3 {a}_{3} {U}_{LO}^{2} \frac{{U}_{RF}}{2} ) & ( 11 ) \\ R ( {I}_{VD 2} - {I}_{VD 3} ) = 2 R ( - {a}_{1} \frac{{U}_{RF}}{2} + 2 {a}_{2} \frac{{U}_{RF}}{2} {U}_{LO} - {a}_{3} \frac{{U}_{RF}^{3}}{2} - 3 {a}_{3} {U}_{LO}^{2} \frac{{U}_{RF}}{2} ) & ( 12 )\end{matrix}$$

Adding equations (11) and (12), the following is obtained:
U_{LOAD}=4Ra₂U_{RF}U_{LO} (13)

From Eq.(13), it is shown that the balance mixer 4 realizes multiplication of received probing pulses U_{RF} with the reference pulses U_{LO}.

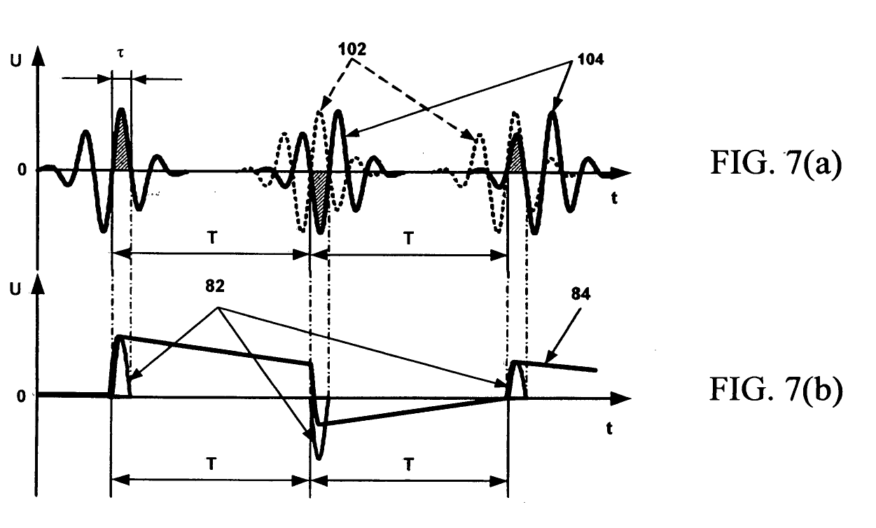

FIG. 7(a) shows the time diagrams of the received probing pulses 104 and the reference pulses 102 at the balance mixer 4, where the received probing pulses 104 are shown by the solid line and the reference pulses 102 are shown by the dashed line. As the arterial blood vessel moves toward and away from the monitoring apparatus, the corresponding time variation of phase difference (FIG. 7(a)) results in positive and negative pulses at the output of the balance mixer 4 (FIG. 7(b).

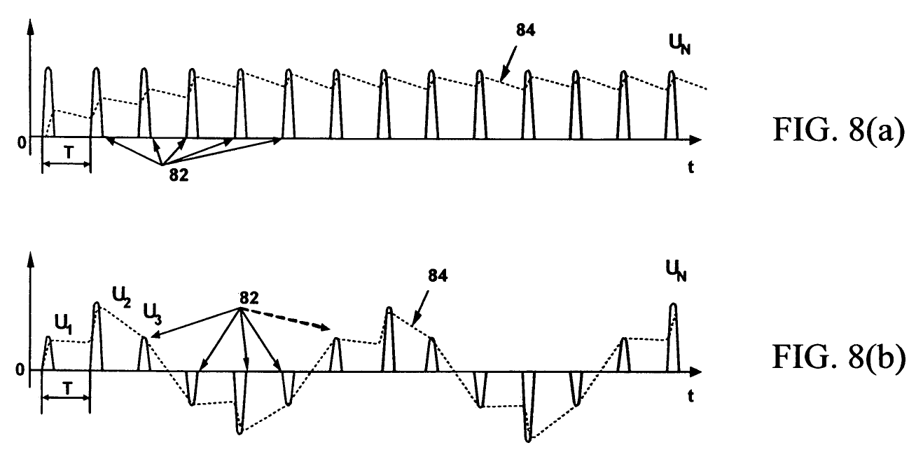

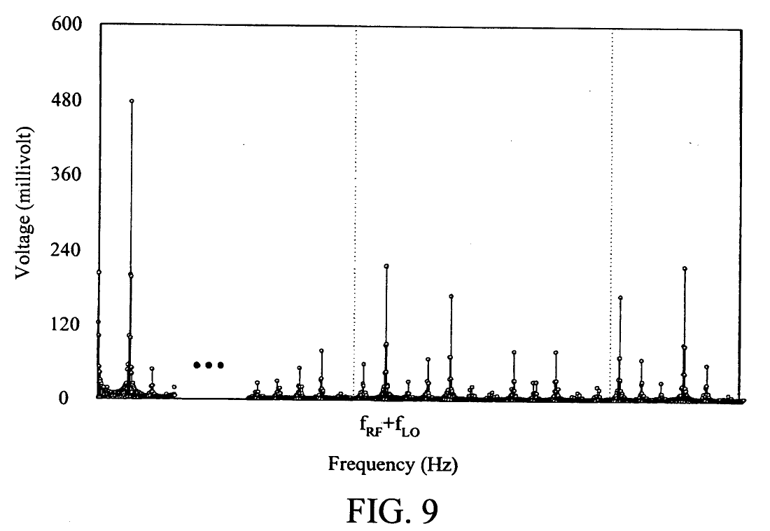

FIG. 8(a) and FIG. 8(b) further illustrate the comparison between the time diagram for the motionless arterial blood vessel and that for the moving arterial blood vessel. The output pulses 82 at the balance mixer 4 then proceed to the first low-pass filter 6 for filtering of undesired high frequency components to generate output signals 84. As shown in FIG. 9, the peaks at frequencies nf_{RF}+mf_{LO} represent repetitive frequency f_{RF} of the received probing pulses and the repetitive frequency f_{LO} of the reference pulses respectively. These high frequency components are filtered-out by the first low-pass filter 6 and the frequency component F, representing the signal modulated by the movement of the arterial blood vessel, is selected.

Application of the balance mixer 4 allows making the operation of multiplication between input and reference pulses more precisely. In other words, a greatly decreasing of output signal distortions of the mixer is obtained. The balanced circuit configuration allows providing a high isolation between input signal (from antenna) and reference input, and between these inputs and the mixer's output (mean of isolation is about 40 dB), that noticeably reduces infiltration of reference pulses to the input of mixer and radiation them by the reception antenna.

Referring back to FIG. 5(a), the high-pass filters 5 consisting of capacitors 41 and 42 removes DC component, which is formed because of scattered signals from the stationary tissue surrounding the blood vessel 26. The first low-pass filter 6, which consists of resistor 38, capacitor 40, resistor 37 and capacitor 39, is used to select low frequency signal corresponding to the movement of the arterial blood vessel 26. The low frequency signal is then amplified by the first amplifier 7, which consists of a first stage amplification (a low-frequency instrumentation amplifier) and a second stage amplification. Gain of the first amplification is set at 117.28 (41.38 dB) by adjusting the resistance of the resistor 51. The first stage amplification also provides suppression of common-mode interference not less than 110 dB.

As shown in FIG. 5(b), the signal enters the second stage of amplification, on basis of operational amplifiers 71 and 72. Gain of the second stage is adjusted by resistors 721 and 722 and is determined by the following formula:

$$K 2 = - [ \frac{R ( 721 )}{R ( 722 )} ] = - [ \frac{100 k Ohm}{10 K Ohm} ] = - 10 ( 20 dB )$$

The resistance of the resistor 723 is determined by the following formula:

$$R ( 723 ) = \frac{R ( 721 ) \cdot R ( 722 )}{R ( 721 ) + R ( 722 )} = \frac{1000}{110} = 9.1 K Ohm$$

Referring to FIG. 5(c), amplified signals enter the second low-pass filter 8 (the fourth-order Butterworth active filter) based on the operational amplifiers 73 and 74. This type of filter provides the most uniform frequency response within the pass band.

Filtered signal goes into the third stage amplification at the second amplifier 9 based on the operational amplifier 75. Gain of the third stage is adjusted by resistors 751 and 752 and is determined by the following formula:

$$K 3 = - ( \frac{R ( 751 )}{R ( 752 )} ) = - ( \frac{4.7 k Ohm}{2 k Ohm} ) = - 2.35 ( 7.4 dB )$$

Total gain of the receiver is equal to:
K=K1K2K3=117.28·10·2.35=2756.08(68.8 dB)

Referring to FIG. 5(d), virtual-ground is created using a voltage divider buffered by an operational amplifier 80. This circuit will generate a virtual-ground reference at ½ of the supply voltage. The circuit includes compensation to allow for bypass capacitors 801, 52, 724, and 753 at the virtual-ground output. The benefit of a large capacitor is that not only does the virtual-ground present a very low DC resistance to the load, but its AC impedance is low as well. The operation amplifier 80 should both sink and source more than 5 mA, which improves recovery time from transients in the load current.

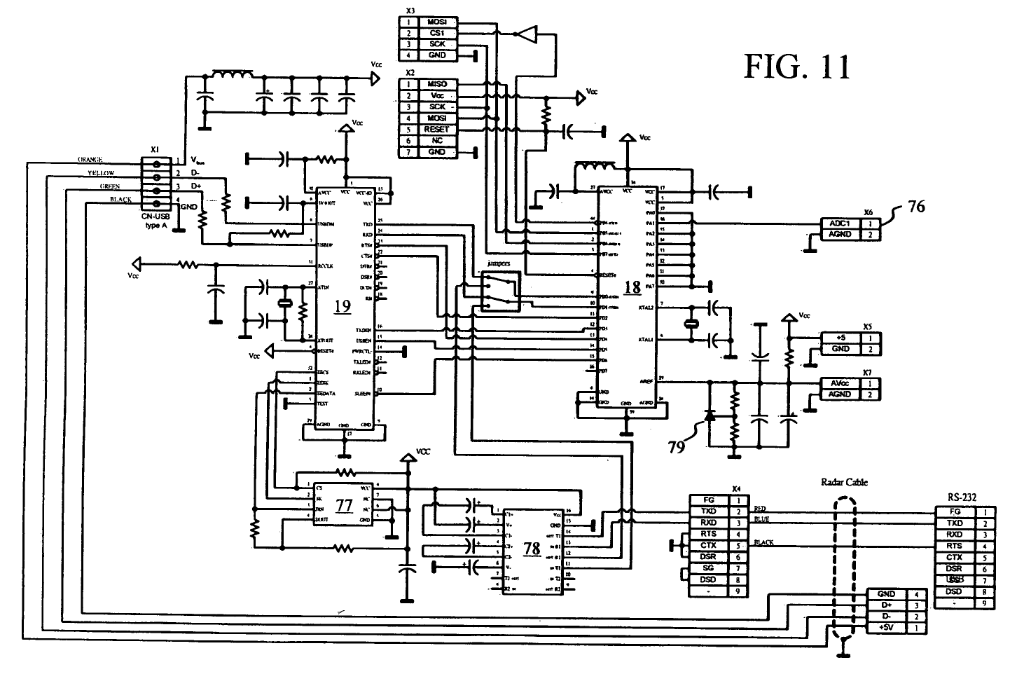

FIG. 11 shows a circuit diagram of the digital board 25 according to the present invention. Amplified, filtered signal is transmitted from the analog board 24 via a connector 76 to an analog-to-digital converter embedded in the microcontroller 18. The microcontroller 18 is preferably a low-power CMOS 8-bit microcontroller based on the RISC architecture. It carries out data collection and data transmission from the analog signal processing circuits. Data transmission can be carried out either by interface RS-232 or by interface USB. Driver of COM-port 78 is used to connect the digital board 25 with an external data processing and display unit 28 by interface RS-232. The transceiver 19 and memory EEPROM 77 are used for communication via interface USB. The transceiver 19 is a single chip USB UART (U-UART) for transferring serial data over USB with a data transfer rates up to 920 k baud. The EEPROM 77 required for storage of the configurable parameters includes the USB Vendor ID (VID), Product ID (PID), Serial Number and Strings of the controller. Source of the reference voltage on the basis of three-terminal adjustable shunt regulator 79 is installed to supply analog signal processing circuits of the analog board 23 and generation of reference voltage of microcontroller's ADC in the digital board 25.

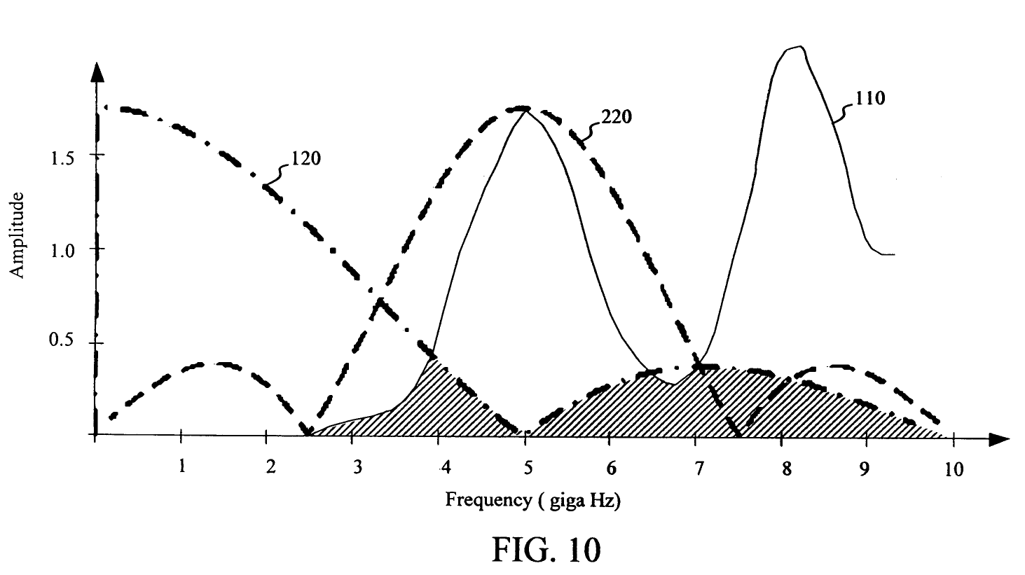

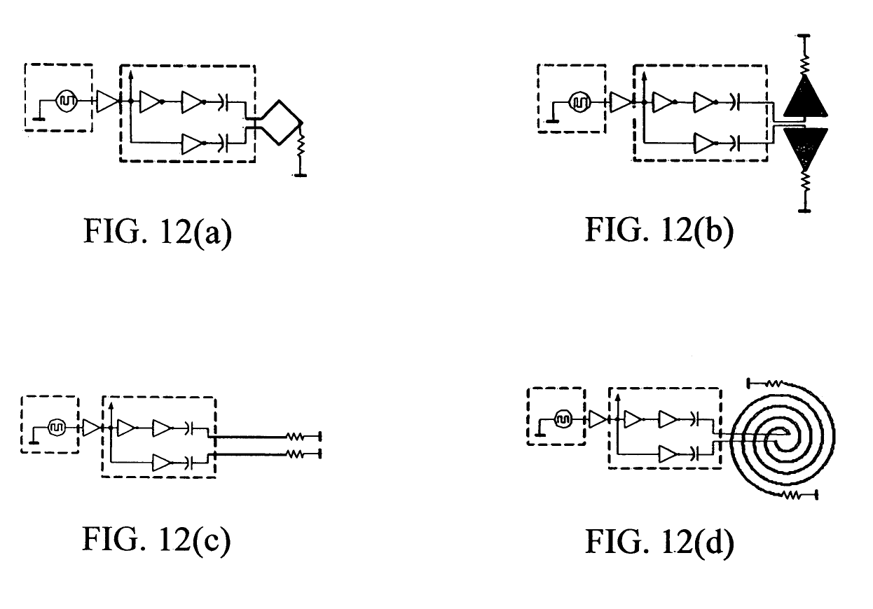

Referring to FIG. 10, improvement in the energy performance of the present monitoring apparatus 10 is due to matching between the signal amplitude spectrum 220 into the transmission antenna 21 and the frequency performance 110 of the transmission antenna 21. As a result, the radiated signal energy is nearly twice as much as that of the conventional device with signal spectrum envelope 120 and identical antenna frequency performance. Matching between signal amplitude spectrum and antenna's frequency performance can be achieved by a suitable selection of transmission antennas such as loop antenna, a bow-tie antenna, a terminating half-wave antenna and a spiral antenna, as shown in FIGS. 12(a), 12(b), 12(c) and 12(d), respectively.

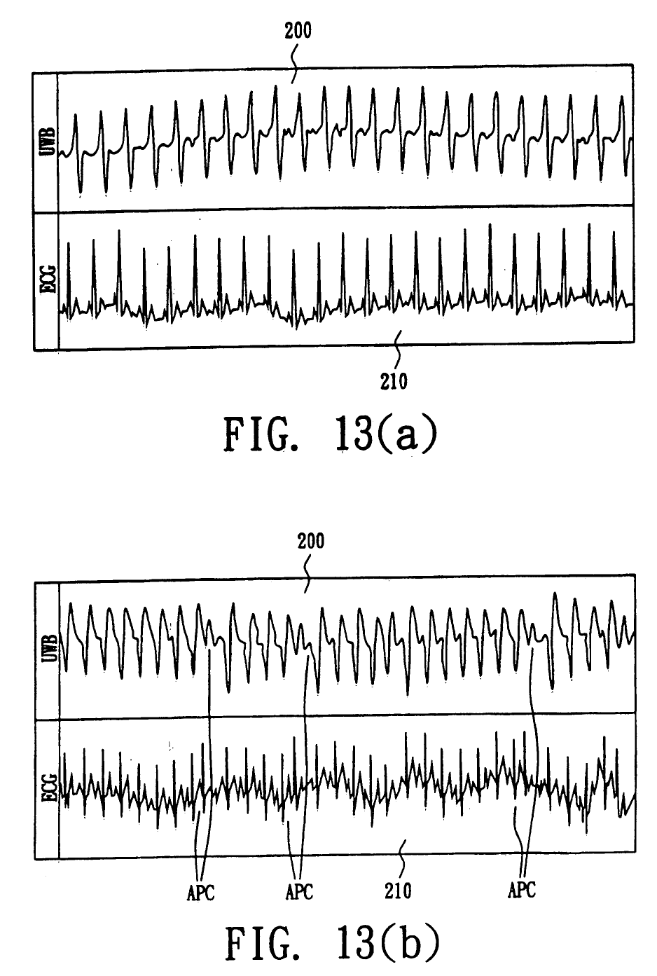

FIGS. 13(a) and FIG. 13(b) show the comparison of radial arterial signal waveform using the present monitoring apparatus and the electrocardiogram signal waveform. As shown in FIG. 13(a) and FIG. 13(b), the performance of the present non-contact arterial monitoring apparatus was verified in the clinical setting. The subject's radial artery pulse signal 200, obtained using the present monitoring apparatus, was compared to the subject's electrocardiogram 210. The result in FIG. 13(a) was obtained from a patient with normal heart rhythm, whereas that in FIG. 13(b) was obtained from a patient with symptom of arterial premature contraction (APC). Both results show excellent beat-to-beat match indicating that the monitoring apparatus can be used as a diagnostic tool for identification of patients with heart diseases.

The above-described embodiments of the present invention are intended to be illustrative only. Numerous alternative embodiments may be devised by those skilled in the art without departing from the scope of the following claims.

1. A monitoring apparatus of arterial pulses, comprising:
an antenna board being comprised of:
a transmission antenna radiating ultra wideband electromagnetic pulses to an arterial blood vessel; and
a reception antenna for receiving the ultra wideband electromagnetic pulses scattered by the arterial blood vessel;
an analog board with a plurality of electronic devices acquiring analog signals representing the arterial pluses of the arterial blood vessel;
a digital board with a plurality of electronic devices digitalizing the analog signal; and
a display device showing the arterial pulses.

2. The monitoring apparatus of arterial pulses according to claim 1, wherein said transmission antenna and the reception antenna are selected from the group consisting of a loop antenna, a bow-tie antenna, a terminating half-wave antenna and a spiral antenna.

3. The monitoring apparatus of arterial pulses according to claim 2, wherein the analog board comprises:
a balance mixer electrically connected to the reception antenna;
at least one filter electrically connected to the balance mixer; and
at least one amplifier electrically connected to the filter.

4. The monitoring apparatus of arterial pulses according to claim 3, wherein the analog board comprises:
a high pass filter electrically connected to the balance mixer;
a first low pass filter electrically connected to the high pass filter;
a first amplifier electrically connected to the first low pass filter;
a second low pass filter electrically connected to the first amplifier; and
a second amplifier electrically connected to the second low pass filter.

5. The monitoring apparatus of arterial pulses according to claim 4, wherein the first amplifier is comprised of an instrumentation amplifier.

6. The monitoring apparatus of arterial pulses according to claim 4, wherein the second low pass filter is comprised of a fourth-order active low-pass Butterworth filter.

7. The monitoring apparatus of arterial pulses according to claim 3, wherein the analog board further comprises:
a clock generator;
a delay line electrically connected to the clock generator;
a first pulse generator electrically connected to the delay line and the balance mixer; and
a second pulse generator electrically connected to the clock generator and the transmission antenna.

8. The monitoring apparatus of arterial pulses according to claim 1, wherein the digital board comprises a microcontroller with an embedded analog-to-digital converter converting the analog signal into a digital signal.

9. The monitoring apparatus of arterial pulses according to claim 8, wherein the digital board further comprises a transceiver electrically connected to the microcontroller transferring serial data to the display device via a universal serial bus.

10. The monitoring apparatus of arterial pulses according to claim 8, wherein the digital board further comprises a COM port driver electrically connected to the microcontroller transferring data to the display device using an RS232 cable.

11. A method for acquiring arterial pulses, said method comprising the following steps:
radiating ultra wideband electromagnetic pulses to an arterial blood vessel;
measuring phase difference between the ultra wideband electromagnetic pulses scattered by the arterial blood vessel and reference ultra wideband electromagnetic pulses; and
acquiring the arterial pulses based on the phase difference.

12. The method for acquiring arterial pulses according to claim 11, wherein said step of measuring phase difference is according to the following equation:
$$Δ φ = Δ φ 1 - Δ φ 2 = \frac{4 π \cdot \sqrt{ɛ \cdot } f}{c} ( {R}_{1} - {R}_{2} )$$
$${R}_{2} = {R}_{1} - V {T}_{a}$$
wherein R1 represents a distance between the arterial blood vessel and where the ultra wideband electromagnetic wave is radiated, V represents a radial velocity of the arterial blood vessel toward where the ultra wideband electromagnetic wave is radiated, Ta represents a time interval as the arterial blood vessel moves from R₁ to R₂, f represents the frequency of the ultra wideband electromagnetic wave, and c represents velocity of light.
